(19) United States
(12) Patent Application Publication
(10) Pub. No.: US 2010/0009914 A1
(43) Pub. Date: Jan. 14, 2010

(54) METHOD OF PROTECTING SENSITIVE MOLECULES FROM A PHOTO-POLYMERIZING ENVIRONMENT

(75) Inventors: Bianca Baroli, Cagliari (IT); Robert Langer, Newton, MA (US); Venkatram Prasad Shastri, Lower Gwynedd, PA (US)

Correspondence Address: CHOATE, HALL & STEWART LLP, TWO INTERNATIONAL PLACE, BOSTON, MA, 02110, US

(73) Assignee: Massachusetts Institute of Technology, Cambridge, MA (US)

(21) Appl. No.: 12/496,680

(22) Filed: Jul. 2, 2009

Related U.S. Application Data
(63) Continuation of application No. 10/757,632, filed on Jan. 14, 2004.

(51) Int. Cl. A61K 38/16; A61P 43/00

(52) U.S. Cl. 514/12

(57) ABSTRACT

In one embodiment, the present invention is a substrate system of photo-polymerizable monomers and bioactive molecules admixed with the monomers and shielded from the monomers by an insoluble material that undergoes a solid-gel transition at body temperature. Upon polymerization, the monomers produce a cross-linked structure and the shielded bioactive molecules are protected from attack in the polymerized environment. In different aspects, the substrate system is used for drug delivery and tissue engineering and protection of enzymes, proteins and growth factors. In another embodiment, the present invention is a drug delivery system of photo-polymerizable monomers, drug molecules associated with the monomers and shielded from the monomers by an insoluble material that undergoes a solid-gel transition at body temperature, and a photopolymerizing means for polymerizing the monomers to produce a cross-linked structure including the drug molecules.

### FIELD OF INVENTION

The present invention is directed towards a method of protecting drugs from damage during polymerization. More specifically, the present invention relates to covering drugs with a temporary shield in such a way that they are not accessible to degradative or denaturing environments during the polymerization process.

### BACKGROUND OF THE INVENTION

In recent years, monomers that are polymerizable upon exposure to light radiation have been explored as starting materials for the production of three-dimensional matrices. These matrices have the potential advantage of being formed in-vivo at the tissue site of interest via minimally invasive procedures, and can be used as scaffolds in tissue engineering, for cell encapsulation, as drug delivery systems, and as fillers for a tissue defect. See Muggli D S, Burkoth A K, Keyser S A, Lee H R, Anseth K S, “Reaction behavior of biodegradable, photo-cross-linkable polyanhydrides,” Macromolecules 3, 4120-4125 (1998); Lu S, Anseth K S, “Photopolymerization of multilaminated poly(HEMA) hydrogel for controlled release,” J Controlled Release 57, 291-300 (1999); Elisseeff J, Anseth K, Sims D, McIntosh W, Randolph M, Langer R, “Transdermal photopolymerization for minimally invasive implantation,” Proc Natl Acad Sci USA 96(6), 3104-3107 (1999); Burkoth A K, Anseth K S, “A review of photo-crosslinked polyanhydrides: In situ forming degradable networks,” Biomaterials 21(23), 2395-2404 (2000); Elisseeff J, McIntosh W, Anseth K, Riley S, Ragan P, Langer R, “Photoencapsulation of chondrocytes in poly(ethyleneoxide)-based semi-interpenetrating networks,” J Biomed Mater Res 51(2), 164-171 (2000); Cruise G M, Hegre O D, Lamberti F V, Hager S R, Hill R, Scharp D S, Hubbel J A, “In vitro and in vivo performance of porcine islets encapsulated in interfacially photopolymerized poly(ethylene glycol) diacrylate membranes,” Cell Transplant 8(3), 293-306 (2000); Smeds K A, Grinstaff M W, “Photocrosslinkable polysaccharides for in situ hydrogel formation,” J Biomed Mat Res 54(1), 115-121 (2001); all incorporated herein by reference.

Although significant advancements have been made in photopolymerization in a biological environment, the concern as to whether the polymerizing environment could be deleterious to sensitive or reactive molecules, which are entrapped within the matrix, remains to be addressed. In addition to possible light-induced alterations such as photo-oxidation, sensitive molecules may be chemically altered upon reacting with monomers, matrix components, and polymerizing species. See Davies M J, Truscott R J W, “Photo-oxidation of proteins and its role in cataractogenesis,” J Photochem Photobio B:Biology 63, 114-125 (2001), herein incorporated by reference. Denaturation reactions are of significance, because entrapped drugs may lose their activity or trigger an immune response. See McNally E J, editor, “Protein formulation and delivery,” New York:Marcell Dekker, Inc. (2000); Cleland J L, Powell M F, Shire S J, “The development of stable protein formulations: A close look at protein aggregation, deamination, and oxidation,” Crit Rev Ther Drug Carrier Syst 10(4), 307-377 (1993); all herein incorporated by reference. Although some studies have shown that proteins can be released from photo-polymerized matrices, there are few reports of enzyme entrapment. See Mellot M B, Searchy C, Pishko M V, “Release of protein from highly cross-linked hydrogels of poly(ethylene glycol)diacrylate fabricated by UV polymerization,” Biomaterials 22, 929-941 (2001); Elisseeff J, McIntosh W, Anseth K, Langer R, “Cogelation of hydrolysable cross-linkers and poly(ethylene oxide) dimethacrylate and their use as controlled release vehicles,” in Dinh S M, DeNuzzio J D, Comfort A R, editors, “Intelligent materials for controlled release,” Washington D.C.:ACS, 1-13 (1999); An Y, Hubbell J A, “Intraarterial protein delivery via intimally-adherent bilayer hydrogels,” J Controlled Release 64, 205-215 (2000); Elisseeff J, McIntosh W, Fu K, Blunk T, Langer R, “Controlled-release of IGF-I and TGF-β1 in a photopolymerizing hydrogel for cartilage tissue engineering,” J Orthop Res 19(6), 1098-1104 (2001); all herein incorporated by reference. Nevertheless, in these latter cases, no quantitative assessment was made regarding the extent of enzyme inactivation or enzyme structure modification.

Consequently, one aspect of the present invention is to protect drugs with a temporary shield such that they are not accessible to degradative or denaturing environments during the polymerization process.

### SUMMARY OF THE INVENTION

In one aspect, the present invention is a substrate system of photo-polymerizable monomers and bioactive molecules admixed with the monomers and shielded from the monomers by an insoluble material that undergoes a solid-gel transition at body temperature. Upon polymerization, the monomers produce a cross-linked structure and the shielded bioactive molecules are protected from attack in the polymerizing environment. In different embodiments, the substrate system is used for drug delivery and tissue engineering and protection of enzymes, proteins and growth factors. In another aspect, the present invention is a drug delivery system of photo-polymerizable monomers, drug molecules associated with the monomers and shielded from the monomers by an insoluble material that undergoes a solid-gel transition at body temperature, and a photopolymerizing means for polymerizing the monomers to produce a cross-linked structure including the drug molecules.

### BRIEF DESCRIPTION OF THE DRAWING

FIG. 1A is a graph of matrix weight loss as a function of time of the incubation aqueous medium for matrix A, B, and C;

FIG. 1B is a graph of pH variation as a function of time of the incubation aqueous medium for matrix A, B, and C;

FIG. 2 is an E-SEM image of matrix C when it was formulated with unprotected enzymes (A) or protected enzyme (B);

FIG. 3 is a photomicrograph comparing enzyme crystal appearance before and after polymerization;

FIG. 4 is a bar graph showing the enzymatic activity retention of protected and unprotected enzymes after 1 day of diffusion out of 3 mm-thick matrices; and

FIG. 5 is a photomicrograph illustrating retention of shape and opacity of HRP-loaded granules, after exposure to the unpolymerized monomer for 2 days, and subsequent polymerization of the monomer.

### DETAILED DESCRIPTION OF THE PREFERRED EMBODIMENTS

In one aspect, the present invention is a substrate system comprising a photo-polymerizable monomer and bioactive molecules admixed with the monomers. The bioactive molecules are shielded from the monomers by an insoluble material that undergoes a solid-gel transition at body temperature. In one embodiment, the insoluble material is insoluble in the monomer. Upon polymerization, the monomers produce a cross-linked structure and the shielded bioactive molecules are protected from attack in the polymerizing environment.

In one embodiment, the substrate is used for drug delivery. In another embodiment, the substrate is used for tissue engineering. In another embodiment, the substrate is used for diagnostic purposes. In another embodiment, the substrate is used for detoxification/substance removal.

The monomer may belong to any class of compounds, may be of any molecular weight, and may react directly or indirectly to any electromagnetic radiation by polymerizing. In certain embodiments, electromagnetic radiation is comprised under UV, Visible or IR spectrum. When reacting indirectly, a suitable system of one, or a mixture of, photoinitiators and accelerators may be responsible of the radiation energy transfer to the monomer. In certain other embodiments, photoinitiators may include radical polymerization by either photoclevage or hydrogen abstraction, or cationic photopolymerization.

The insoluble material may be a gelatin, collagen, natural polymer or synthetic polymer. The bioactive material may be a drug, enzyme, protein or growth factor.

Where the bioactive material is a drug, the drug may be a calcifying agent, antibiotic, anticancer agent, anti-inflammatory agent, cytokine, matrix metalloproteinase, cell mediator, inhibitor, antimitotic agent, alkylating agent, immunomodulator, antihypertensive, analgesic, antifungal, antibody, vaccine, hormone, cardiovascular agent, respiratory agent, sympathomimetic agent, cholinomimetic agent, adrenergic and adrenergic neuron blocking agent, antimuscarinic and antispaspodic agent, skeletal muscle relaxant, diuretic, uterine and antimigrane agent, local anesthetic, antiepileptics, psicopharmacological agent, histamine and antihistamine, central nervous system stimulants, antineoplastics and immunosuppressive agent, vitamins and other nutrients, antimicrobial agent not comprised in antibiotics, antiviral agent, parasiticides or diagnostic agent. In one embodiment, the drug is bulked up with one or a mixture of compatible substrates. The compatible substrate may be selected from a group consisting of sugars, polysaccharides, glycolipids, glycosaminoglycans, lipids, amino acids (e.g.; but not limited to: glycine, sodium glutamate, proline, α-alanine, β-alanine, lysine-HCl, 4-hydroxyproline), peptides and polypeptides, proteins, amines (e.g.; but not limited to: betaine, trimethylamine N-oxide), lipo-proteic molecules, polyols, gums, waxes, antioxidants, anti-reductants, buffering agents, inorganic and organic salts (e.g; but not limited to: ammonium, sodium, and magnesium sulfate, potassium phosphate, sodium fluoride, sodium acetate, sodium polyethylene, sodium caprylate, propionate, lactate, succinate), radical scavengers, diluents (e.g.; but not limited to: mannitol, lactose, sorbitol, sucrose, inositol, dicalcium phosphate, calcium sulfate, cellulose, hydroxypropylmethylcellulose, kaolin, sodium chloride, starch), cryoprotectants, and natural or synthetic polymers. In another embodiment, the substrate system further includes a binder (e.g.; but not limited to: starch; gelatin; sugars as sucrose, glucose, dextrose, molasses, and lactose; natural and synthetic gums such as acacia, sodium alginate, extract of Irish moss, panwar gum, ghatti gum, mucilage of isapol husks, carboxymethylcellulose, methylcellulose, hydroxypropyl methylcellulose, hydroxylpropyl cellulose, ethyl cellulose, polyvinylpyrrolidone, Veegum, larch arabogalactan; polyethylene glycols; ethylcellulose; waxes; water and achools, amylase, methacrylate and methyl methacrylate copolymers), plasticizer (e.g; but not limited to: glycerin, propylene glycol, polyethylene glycols, triacetin, acetylated monoglyceride, citrate esters, phthalate esters) or disaggregant (e.g.; but not limited to: starches, clays, celluloses, algins, gums, cross-linked natural and synthetic polymers, Veegum HV, methylcellulose, agar, bentonite, cellulose and wood products, natural sponge, cation-exchange resins, alginic acid, guar gum, citrus pulp, carboxymethylcellulose, combinations of sodium lauryl sulfate and starch) used in any of their physical or processed state. Any derivative of above-mentioned molecules are included as well.

The bioactive molecules may be shielded by the insoluble material by granulation, spray drying, spray chilling, lyophilization, coating vapor deposition (CVD), compression, microencapsulation, coating, subcoating, sealing, coacervation, suspension, precipitation, cogelation, gelation, inclusion in pre-formed delivering systems, inclusion into matrix and micromatrix, or evaporation.

In another aspect, the present invention is a substrate system comprising a photo-polymerizable monomer and bioactive molecules, previously included in any drug delivery system. In one embodiment, if the drug loaded delivery system would be unstable in the presence of the non-polymerized monomer, the drug delivery system is protected prior to being introduced into the non-polymerized monomer. The drug-loaded delivery systems are shielded from the monomers by an insoluble material that undergoes a solid-gel transition at body temperature. Drug delivery systems may include, but are not limited to, any type and dimension of: capsules, tablets, powders, granules, pills, pellets, reservoir devices, matrix devices, microparticles or microspheres, nanoparticles or nanospheres, micro- and nano-capsules, liposomes, lyophilized systems, osmotic systems, emulsions, microemulsions, gels, gelified systems, implants, implantable mems, implantable micro- and nano-diagnostic devices, solid lipid nanoparticles, chip, microchips, microarrays, environmental sensitive systems, immune system sensitive systems, dissolution-controlled systems, swellable systems, osmotic pumps and micro-pumps, magnetic systems, ciclodextrins, human or animal and normal or stem or immortalized or engineered cells.

In another aspect, the present invention is a drug delivery system comprising photo-polymerizable monomers, drug molecules and a photopolymerization means for polymerizing the monomers to produce a cross-linked structure including the drug molecules. The drug molecules are associated with the monomers and shielded from the monomers by an insoluble material that undergoes a solid-gel transition at body temperature. Photopolymerization means can include but are not limited to: UV radiation, blue-light and visible radiations, radiations produced by light emitting diodes technology.

### Exemplification:

A study was conducted to show that the photopolymerization step is a source of enzyme alteration for an unprotected enzyme and to compare that result with a photopolymerization step conducted with a protected enzyme. In this study, two sensitive molecules, horseradish peroxidase (HRP) and α-glucosidase (α-GLS), were tested in an unprotected and protected form. The protective formulation was developed based on the use of nonaccessible substances, since the polymerizing environment would not affect nonaccessible substances. Enzymes used in the study were protected by wet granulation, although different techniques may be used and the present invention is not so limited. See Benita S, editor, “Microencapsulation,” Methods and industrial application, New York: Marcel Dekker, Inc. (1996); Remingtong J P, “Remingtong's Pharmaceutical Sciences,” 18^{th} ed., Easton: Mack Publishing Company (1990); all herein incorporated by reference. After entrapment in a photo-cured matrix, enzymes were recovered by passive diffusion and characterized by activity retention and MALDI-TOF analysis. See Pandey A, Mann M, “Proteomics to study genes and genomics,” Nature, 405(6788), 837-846 (2000); Gygi S P, Aebersold R, “Mass spectrometry and proteomics,” Curr Opin Chem Bio 4, 489-494 (2000); all herein incorporated by reference.

### Materials

Horseradish peroxidase (HRP; Lot. AE599921), immunopure® TMB dihydrochloride (TMB: 3,3′,5,5′-tetramethylbenzidine), a stable peroxide solution (10×), and Micro BCA protein Assay Reagent Kit were bought from Pierce (Rockford, Ill.). α-Glucosidase (α-GLS; Lot. 179AB) was ordered from Biozyme Laboratories (Biozyme Laboratories Limited, Blaenavon, South Wales, UK). Sodium hydroxide, sulfuric acid, acetone, and sodium phosphate monobasic were purchased from Mallinckrodt Chemicals (Mallinckrodt Baker, Inc., Phillipsburg, N.J.). Ethyl 4-dimethylaminobenzoate (4-EDMAB), camphorquinone (CQ), 4-hydroxybenzoic acid (4-HBA), 1,6-dibromo hexane (96%) and poly(ethylene glycol)-dimethacrylate (PEGDM; Mₙ ca. 550) were obtained from Aldrich (St. Louis, Mo.); β-lactose, bovine serum albumin (BSA; fraction V) and gelatin A and B from Sigma (St. Louis, Mo.); sodium and potassium phosphates from Fisher Chemicals (Pittsburgh, Pa.); 4-Nitrophenyl-α-D-glucopyranoside (PNPG) from Fluka (St. Louis, Mo.). All chemicals were used as received and stored as specified by the suppliers. 1,6-(Bis-p-carboxyphenoxy)hexane (CPH) was synthesized and characterized as previously described. See Muggli supra.

### Enzyme Formulations

Horseradish peroxidase (HRP) and α-glucosidase (α-GLS) were used as model enzymes. Their unprotected and protected formulations were simple and granulated powders, respectively. These two powders were prepared according to the following procedure.

Each enzyme (E) was first pulverized by trituration on a Teflon solid support with a Teflon-coated spatula. Pulverized enzyme was then intimately mixed with β-lactose (L), in a ratio of 1:100 w/w (E:L), by geometrical dilutions until a homogeneously colored powder was obtained (ca. 15 minutes). This first formulation is referred to herein as the unprotected form. Subsequently, a portion of the unprotected enzyme-lactose mixture was granulated with a 5% aqueous solution of gelatin B to produce a slightly wet mass, which was then forced through a sieve (sieve no. 78, opening 212 μm) to yield granules. The granules were then dried for 1 or 2 h under vacuum, at room temperature, and in absence of light before further use. This granulated power (or granules) is herein referred to as the protected formulation.

Both formulations were stored at 4° C. in a dry atmosphere and analyzed for enzyme activity (sections 2.3 and 2.4) prior to use. To verify the homogeneity of enzyme distribution in the unprotected and protected formulations, freshly prepared powders were subjected to a content uniformity test. See USP 24, “The United States Pharmacopeia 24”, Rockville, Md.: The United States Pharmacopeial Convention, Inc. (2000); herein incorporated by reference. Both formulations were sampled uniformly over their entirety without mixing. The amount of formulated enzyme used in the preparation of the delivering matrices was considered an appropriate sample size. See Table 1.

TABLE 1
Photopolymerized Matrices Composition

Compounds (%)

| Formulation | CPH | Salts^{a} | Enzyme^{b} | PEGDM |
|---|---|---|---|---|
| A | 55.23 | 18.41 | 1.47 | 24.89 |
| B | 36.82 | 36.82 | 1.47 | 24.89 |
| C | 18.41 | 55.23 | 1.47 | 24.89 |

^{a}Na₂HPO₄•7H₂O and NaH₂PO₄•H₂O were combined in a ratio 1:1 w/w.
^{b}Enzymes were used in either unprotected or protected form.
Note:
Photopolymerization was initiated by adding CQ and 4-EDMAB (each 0.74% w/w of the final composition) to PEGDM prior to mix all the other components.

Samples (n=10 per each formulation) were then quantified for enzyme content and activity (see next sections for activity assays). For each set of 10 samples, the mean and the standard deviation was calculated. Requirements of the test were considered met if the amount of enzyme and its activity was within the limits of 85 and 115% of their expected values, and the relative standard deviation (expressed in percentage) was equal or less than 6%. The test was successively performed several times over a 6-month period to verify the physical stability of the formulation and the retention of enzyme activity. Enzyme concentration (also referred to as total protein content) was determined with the Micro BCA protein Assay Reagent Kit.

### HRP Activity Determination

Enzymatic activity was calculated from the amount of oxidized TMB produced in a peroxide containing solution. See Josephy P D, Eling T, Mason R P, “The horseradish peroxidase-catalyzed oxidation of 3,5,3′,5′-tetramethylbenzidine,” J Biol Chem 257(7), 3669-3675 (1982); herein incorporated by reference. The concentration of the oxidized product was measured at 450 nm using a UV-visible spectrophotometer (Cary 50 Bio, Varian, Palo Alto, Calif.) (detection limit: 2.0 ng/mL). The assay was adapted to the enzyme concentrations used in this study and performed by mixing 900 μL of stable peroxide substrate buffer (1×) with 900 μL of a TMB aqueous solution (0.4 mg/mL) in disposable polystyrene cuvettes (VWR Scientific Products, Willard, Ohio). Finally, 200 μL of the enzyme solution was added, and absorbance was recorded after 1 minute.

### α-GLS Activity Determination

α-GLS activity was calculated from the amount of p-nitrophenol (PNP) released from PNPG and measured spectrophotometrically at 400 nm. The standard activity assay for α-GLS was modified so that it could be carried out in a 96-well plate. See Bergmeyer H U, editor “Methods of enzymatic analysis,” 2^{nd} ed., New York: Academic Press Inc., Vol. 1, p 459 (1974); herein incorporated by reference. Three buffer solutions were prepared for this assay: (a) a 0.1 M potassium phosphate buffer pH 7.0 (K-PBS 0.1 M), obtained by mixing 650 mL of K₂HPO₄ 0.1 M and 500 mL of KH₂PO₄ 0.1 M; (b) an albumin supplemented buffer (K-PBS-Alb), obtained by adding BSA to K-PBS 0.1 M to a final concentration of 1 g/L; (c) an enzyme dilution buffer (K-PBS 0.01M), prepared by diluting (1:10) K-PBS 0.1 M with Milli-Q water. The K-PBS 0.01 M and the substrate solution (PNPG, 20 mM in Milli-Q water) were kept on ice for at least 2 hours before use. The assay was performed in 96-well plates (Corning, Inc., New York, N.Y.) to which solutions were added in the following order. First, 50 μL of K-PBS 0.01 M, which contained the enzyme to be tested, were pipetted into the well. When required, serial dilutions were directly performed in the 96-well plate with K-PBS 0.01 M, using a multichannel pipettor (VWR Scientific Products, Willard, Ohio). Subsequently, 100 μL of the K-PBS-Alb were added, and the reaction was started upon addition of 50 μL of the substrate solution. Plates were covered with ImmunoWare™ sealing tape (Pierce, Rockford, Ill.) and incubated at 37° C. (Incubator model 1555; Sheldon MFG, Inc., Cornelius, Oreg.). The formation of PNP was detected spectrophotometrically at 400 nm using a 96-well plate reader (Dynatech MR5000; Dynatech Laboratories, Inc., Chantilly, Va.). Calibration curves maintained their linearity over a 24-hour incubation period. Using this procedure, the detection limit of α-GLS was 9 ng/mL and 0.5 ng/mL after 2 and 24 hour of incubation, respectively. Gelatin B and β-lactose were tested for cross-reactivity.

### Three Dimensional Matrix Preparation

Three-dimensional matrices containing protected and unprotected enzymes were prepared by light-induced polymerization of various formulations. See Table 1. First, 4-EDMAB and CQ (0.74% w/w each) were dissolved in the PEGDM monomer. The remaining components were then suspended in PEGDM, and mixed in for 15 minutes, at which time a homogeneous whitish putty-like mass was obtained. Finally, enzyme, in its unprotected or protected form, was added to this putty mass. The mixture was mixed thoroughly for a further minute, and then poured into a cylindrical Teflon mold. Matrix polymerization was achieved by irradiation with blue light (3M Curinglight XL 1500, 420-500 nm, output 400 mW/cm² at a distance of 3 mm, 3M Health Care, USA) for 5 min on either face of the cylindrical matrix. The polymerized matrices were removed from the molds, weighed, and stored in a dry box at room temperature until use, generally for 2 to 3 hours. Matrices were 5 mm in diameter and 3 mm in height. Porosity in three-dimensional matrices was achieved by dissolution of soluble components during enzyme diffusion. Three-dimensional matrices not loaded with the model enzymes were prepared as described above, with the exception that both the unprotected and the protected enzyme formulations were respectively substituted with an equal amount of β-lactose alone.

### Matrix Characterization

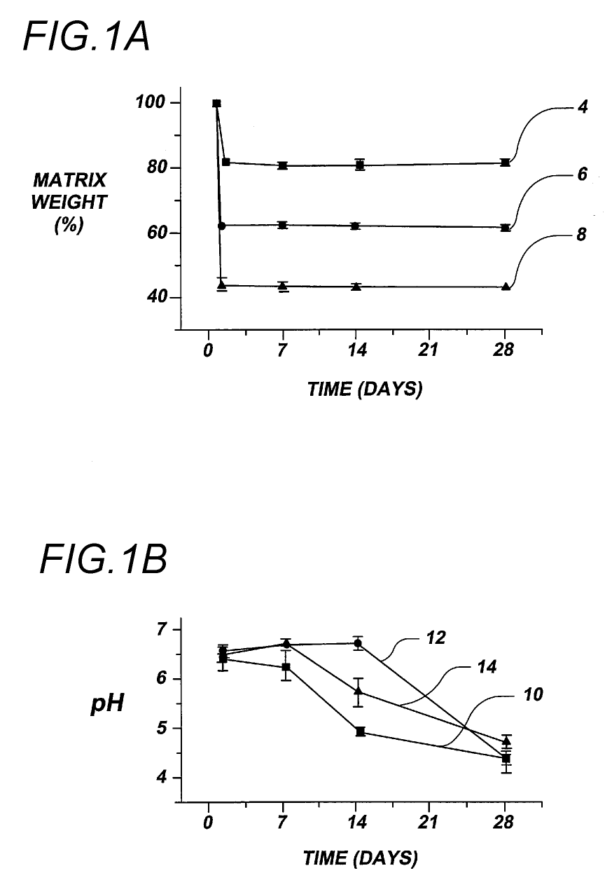

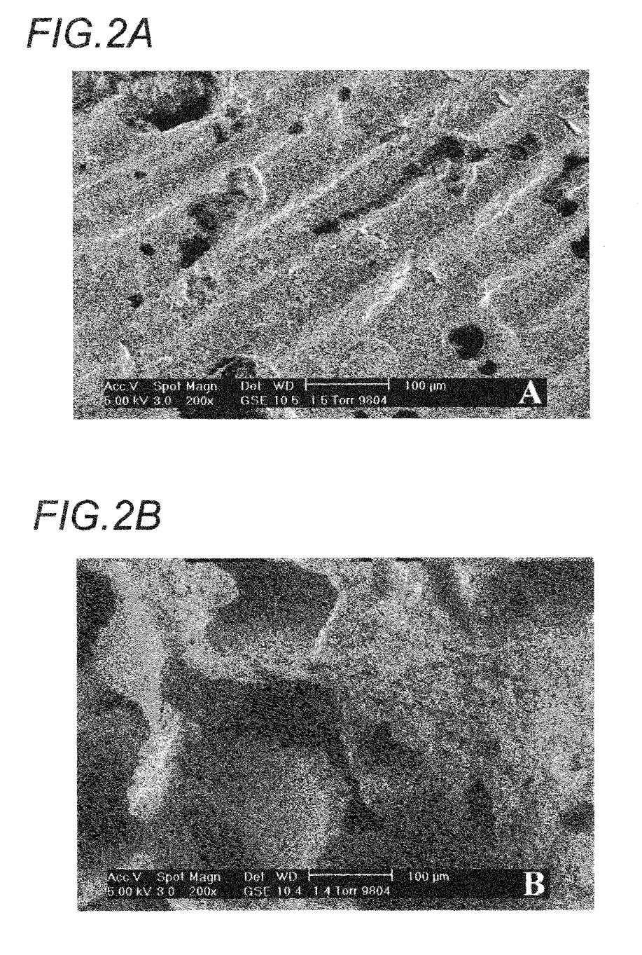

Three-dimensional matrices were characterized for their ability to release compounds that could interfere with the activity and the total protein assays. Specifically, activity assays are sensitive to variations in pH, and total content assays might be sensitive to other species present in the samples to be tested. Weight loss of matrices was studied at 37° C. over a 1-month period and sampled weekly. Samples (Table 1; n=6) were kept in Milli-Q water (4 mL) on an orbital shaker (80 RPM, Bellco Glass, Inc., Vineland, N.J.), and at each time point, they were submitted to the following procedure: the aqueous solution was removed and its pH was measured (pH Meter 430, Corning, Corning, Inc., New York, N.Y.). Matrices were briefly wiped, to remove the excess of water, and then stored in a dry box, under vacuum, until constant weight. Afterward, matrices were weighed, and the changes in weight reported as percentage loss of weight. FIG. 1A is a graphical representation of the data for matrix A (squares) 4, matrix B (circles) 6 and matrix C (triangles) 8. FIG. 1B shows the pH variations of the aqueous solution in contact with matrices as measured for matrix A (squares) 10, matrix B (circles) 12 and matrix C (triangles) 14. The study was then repeated (n=3) under the conditions of the enzymatic activity retention studies to evaluate if the pH of the buffers used in these further studies could be maintained constant. See below. Finally, matrices were imaged by environmental scanning electron microscopy (E-SEM; FEI/Philips XL 30 FEG, FEI Company, Hillsbore, Oreg.). FIG. 2 depicts E-SEM imaging of matrix C when it was formulated with unprotected enzymes (A) or protected enzyme (B).

### Retention of Enzymatic Activity

HRP and α-GLS were used as model molecules to study whether enzymes diffuse through photo-polymerized matrices (n=6) in their active forms, after exposure to the polymerizing environment. The matrices investigated (A, B, and C) were formulated to contain the model enzymes either in their unprotected or protected forms. To prevent protein adhesion, low-binding polypropylene supplies were utilized. Studies were conducted at the temperature that favors the long-term maintenance of enzyme activity and in their specific activity assay buffers (1 mL): PBS (pH: 7.4) at 37° C. for HRP and K-PBS 0.01 M (pH: 7.0) at 4° C. for α-GLS. The incubation medium was completely sampled and vials replenished with fresh buffer every day for the first 5 days, and then on a weekly basis, for 4 weeks thereafter. Sampled solutions were used to determine the total amount of enzyme diffused and its activity, as described in earlier. Activity retention (A.R.) is defined as the ratio of the observed (O.A.) versus expected enzyme activity (E.A.) and it is expressed in percentage.

A.R.=(O.A.)×100/(E.A.)

Activity loss (A.L.) is the difference between expected and retained enzymatic activity; both E.A. and A.R. are expressed in percentage.

A.L.=100−A.R.

The amount of enzyme that was recovered at each time point was used to determine the expected activity from an activity calibration curve of unaffected enzyme.

### Enzymes Characterization by MALDI-TOF Spectrometry

The molecular weight of the enzymes studied was analyzed by MALDI-TOF spectrometry. Enzymes were investigated in three conditions: (1) not formulated (native forms), (2) formulated, and (3) after being released from the photopolymerized matrices. To record finest spectra, samples were extensively purified by dialysis across a Spectra/por 2 membrane (Spectrum Laboratories, Inc., Rancho Dominguez, Calif., mol.wt. cutoff 12-14 kDa) in Milli-Q water for 3 days at 4° C. in the absence of light, and then dried using a SpeedVac concentrator (Savant SVC 200H, Savant Instruments, Inc., Holbrook, N.Y.) for 2 or 3 hours at room temperature. Dried samples were then mixed with a few microliters of sinapinic acid solution (10 mg/mL; Acetonitrile: 0.1% TFA, 30:70 V/V), and analyzed by a Maldi Voyager-DE™ STR (PerSeptive Biosystems Inc., Framingham, Mass.) spectrometer. Spectra of excipients were used as controls, while spectra of dialyzed native enzymes helped to assess if dialysis caused enzymatic alterations.

### Statistic Analysis

Data was analyzed by ANOVA and Student's t-tests; a p of ≦0.05 was considered significant.

### Results

### Enzyme Formulations

Unprotected and protected enzyme formulations were prepared by trituration, and wet granulation, respectively. For both enzymes, 15 minutes of trituration appeared to be sufficient to uniformly distribute the enzyme in the excipient (β-lactose) because the content uniformity test was fulfilled immediately after preparation and over a 6-month storage period. See USP 24 supra. These results indicated that no phase separation occurred between the two powder components (β-lactose and enzyme) during preparation and storage. The enzyme content in the tested samples (n=10) was always within the acceptable range of the 85-115% limits.

The formulative process appeared to be mild as no significant variation (p>0.05) in the enzymatic activity was observed before and after formulation and during the 6 months of observation, compared with nonformulated enzymes stored under identical conditions. In addition, neither bacterial nor fungal colonies were detected in the studied preparations as ascertained by ocular and microscopic examination. Finally, a pilot protected formulation was produced using gelatin A: no significant differences in retention of enzyme activity were observed between samples formulated with gelatin A or gelatin B (unpublished results).

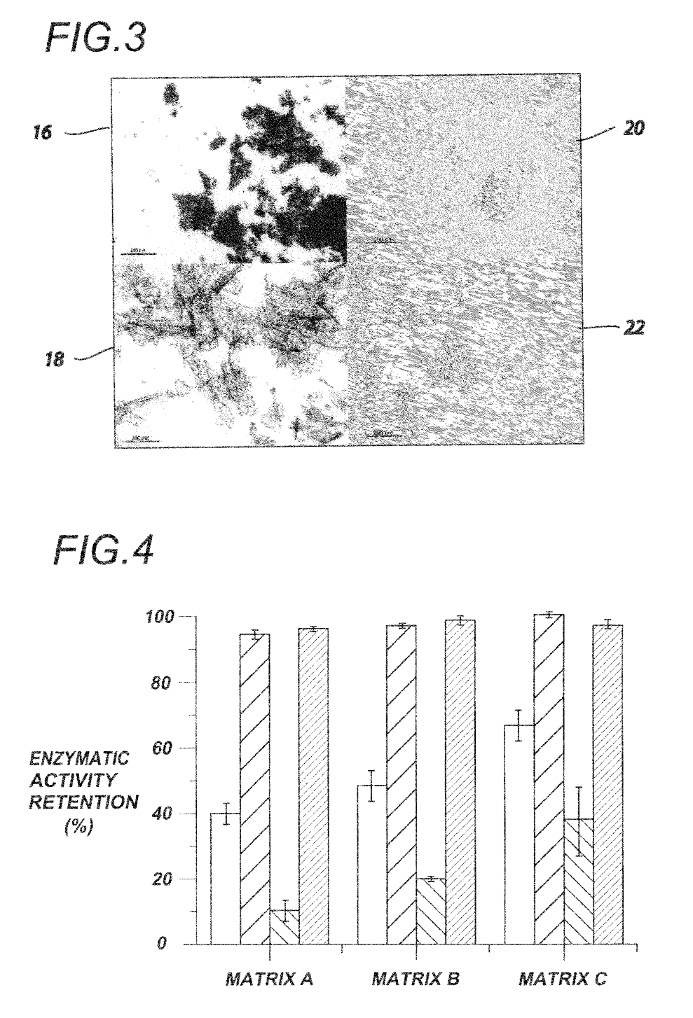

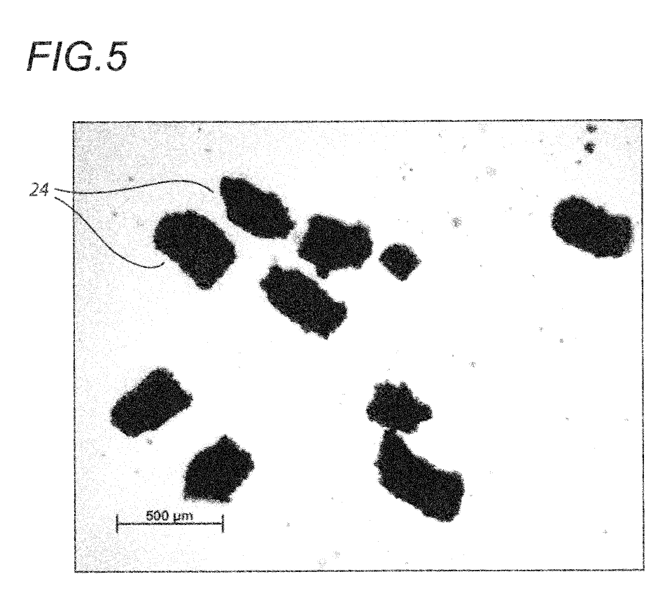

Both formulations dissolved in the activity buffer solutions within a few minutes of contact without agitation. In addition, it was observed, by microscopy, that enzyme crystals (the unprotected formulation) slightly dissolved in the monomer PEGDM after 10 minutes at room temperature, in the absence of light and photoinitiators. FIG. 3 shows a comparison of enzyme crystal appearance before (α-GLS, top left 16; HRP, bottom left 18) and after polymerization (α-GLS, top right 20; HRP, bottom right 22). Pictures in FIG. 3 were acquired by microscopical imaging in differential interference contrast (DIC) using Zeiss Axiovert 200 (5×, Carl Zeiss Microimaging Inc., Thornwood, N.Y.). In contrast, β-lactose and granules did not dissolve (2 days of observation) under the same conditions, and no enzyme leakage from the granules was observed before or after polymerization of the monomer. FIG. 5 shows the retention of shape and opacity of HRP-loaded granules 24, after exposure to the unpolymerized monomer for 2 days, and subsequent polymerization of the monomer. The absence of any brownish shadow around granules is evident, showing that HRP was unable to diffuse in the surrounding monomer before or during polymerization. The picture of FIG. 5 was obtained by microscopical imaging in differential interference contrast (DIC) using a Zeiss Axiovert 200 (5×; Carl Zeiss Microimaging Inc., Thornwood, N.Y.).

### Matrix Preparation and Characterization

Three-dimensional porous matrices were produced by a combined photopolymerization-salt leaching technique. Photopolymerization is a well-understood free-radical process. See Cook W D, “Photopolymerization kinetics of dimethacrylates using the camphorquinone/amine initiator system” Polymer 33(2), 600-609 (1992); herein incorporated by reference. Upon irradiation, CQ is promoted into an excited state (triplet) that dissociates to yield a radical species, reaction that is accelerated in the presence of 4-EDMAB, and consequently initiates the polymerization of PEGDM. CPH was used to add rigidity to the matrix and to prevent shrinkage upon polymerization. It was observed that polymerized matrices continued to maintain their initial dimensions (d: 5 mm; h: 3 mm) upon hydration. To limit interference with the total protein assay (micro-BCA), matrices that do not degrade in the experimental time frame were used. For each formulation, no statistical differences (p>0.05) in mass loss were found between matrices formulated with either unprotected or protected enzyme. In addition, the mass loss observed in the first day appeared to be primarily due to the leaching of soluble components with subsequent formation of matrix porosity. See FIG. 1A and Table 1. Over a 4-week period, an increase in acidity (drop of over 2 pH units) was observed if water was used as the incubation medium. See FIG. 1B. Although matrices showed neither degradation nor fracture formation at a macroscopic level, the observed increased acidity in FIG. 1B could be due to the hydrolysis of PEGDM ester bonds, which link the poly(ethylene glycol) chains to the polymethacrylic chains formed during the photopolymerization. Nevertheless, no such variation in pH was observed when the same experiments were repeated in the activity assay buffers, indicating that the enzyme activity assay itself would not be compromised under the same conditions. Finally, E-SEM imaging showed a higher porosity in the matrices that had the protected enzyme. See FIG. 2.

### Retention of Enzymatic Activity

In the initial studies, both protected and unprotected enzymes (n=6) were suspended in pure PEGDM, and the resulting mixture cured for five minutes into films of 0.2-mm thickness between two Teflon sheets. Due to the low thickness and the transparency of these films, 5 minutes were sufficient to achieve curing. Diffusion of enzymes from films was monitored for 3 days. Over the course of the first 24 hours, 95-100% of the entrapped enzymes were recovered, based on a micro-BCA assay. However, unprotected HRP and α-GLS retained only 31.4+2.3% and 49.9+1.7% of their activity, respectively. In contrast, protected enzymes were recovered with complete retention of activity. The loss in enzymatic activity in the recovered unprotected enzymes are attributed to a negative effect of the polymerizing environment because native (not formulated) enzymes maintained under the same conditions (activity assay buffers and temperature) of these initial experiments did not show an activity loss. These results showed that the polymerizing environment could be capable of inducing changes in enzyme activity, depending on the sensitivity of the molecule being entrapped. These results were also confirmed by MALDITOF analysis. See Table 2.

TABLE 2
MALDI-TOF Molecular Weight Analysis

| | Molecular | Molecular Weight |
|---|---|---|
| Formulation | Weight (Da) | Retention^{a} (%) |
| HRP native forms | 43146.82 | 100.0 |
| HRP unprotected | 43039.53 | 99.75 |
| HRP protected | 43144.63 | 99.99 |
| α-GLS native forms | 68340.60 | 100.0 |
| α-GLS unprotected | 65387.85 | 95.68 |
| α-GLS protected | 68407.24 | 100.09 |

^{a}Molecular weight retention values have an error of 0.01%.

Subsequently, considering that scaffolds used for tissue engineering purposes are often three-dimensional and porous, 3-mm thick matrices, wherein porosity was introduced in situ by dissolution of the soluble salt phase, were employed in further experiments. See Table 1. As in the case of 0.2-mm thick films, protected enzymes retained their activity better than unprotected enzymes. In FIG. 4, enzymatic activity retention of protected and unprotected enzymes after 1 day of diffusion out of 3 mm-thick matrices is shown. Each group of columns is ordered from left to right as follows: unprotected α-GLS, protected α-GLS, unprotected HRP, and protected HRP. The greatest difference in activity between protected and unprotected enzymes was observed during the initial 24-hour period of enzyme diffusion. In particular, the activity of both protected HRP and α-GLS was over 94% with no significant differences (p>0.05) in retention of enzymatic activity between the two enzymes and between the different matrix compositions studied. See Table 1. In contrast, the activity of unprotected enzymes varied greatly and showed a matrix-composition dependence. A trend of increasing activity was observed in formulations with increasing salt and decreasing CPH content. As shown in FIG. 4, the activity of the unprotected HRP remained below 38.3±9.6%, while the activity of unprotected α-GLS ranged between 40.7±3.6% and 66.2±5.0%. Beyond the initial 24-hour period the differences in the retention of enzymatic activity between unprotected and protected enzyme diminished to a maximum of around 5.0±1.4%.

### Enzyme Characterization

Enzymes were analyzed as supplied (not formulated; in their native forms), formulated in their unprotected and protected forms, and after being entrapped and then released from the photopolymerized matrices using MALDI-TOF spectrometry. See Table 2. MALDI-TOF is an extremely sensitive tool to analyze changes in mass of molecules possessing high molecular weights. Changes in mass of 0.01% could be detected in a reproducible manner and represent the sensitivity of the method. The molecular weight of both unprotected HRP and α-GLS, respectively decreased by 0.25% and 4.32%, upon exposure to the polymerizing environment. In contrast, enzymes protected by gelatin-based wet granulation prior to entrapment in PEGDM matrices showed lower changes in mass. The molecular weight of HRP decreased by 0.01% while that of α-GLS increased by 0.09%. These small percent changes in molecular weight translate into differences in mass ranging from 2.19 Da (HRP-protected) to 2952.75 Da (α-GLS-unprotected). It is important to note that a loss in molecular weight by 0.25% and 4.32% corresponded to an activity loss of 68.6±2.3% and 50.1±1.7% for the unprotected formulations of HRP and α-GLS, respectively. These results suggested that even minor changes in the molecular weight of an enzyme could be detrimental to its function. Molecular weights reported in Table 2 are absolute and not averaged because no changes in values were observed upon repeated measurements. No changes in molecular weight were observed between the native and dialyzed enzymes either (data not shown).

### Discussion

HRP and α-GLS were chosen as model drugs because they possess different physiochemical characteristics. HRP is a protein of 305 amino acids (AA), which is positively charged at neutral pH. HRP is characterized by the presence of four disulfide bonds, seven N-linked carbohydrate residues, one pyrrolidone residue, and one heme group. In contrast, α-GLS is an enzyme of 548 AA, which is negatively charged at neutral pH and does not have disulfide bridges. See ExPASy Molecular Biology Server, Home page, http://www.expasy.ch (4, Oct. 2001); herein incorporated by reference. In addition, these enzymes were chosen because (1) their activity is based on a single-step self-catalyzed reaction, and hence, any changes in enzyme kinetics can be directly attributed to alterations of the enzyme structure, and (2) their absorption spectra and thermal sensitivity are different, with HRP absorbing in visible light (due to its prosthetic group) and α-GLS being thermally sensitive.

The design of a protective shield was developed based on the following four considerations. First, the process should be mild: organic solvents and high shear forces should be preferably avoided to minimize alteration to enzyme structure during formulation. Second, excipients, binders and compounds used for formulate enzymes should be insoluble in the monomer (PEGDM) to impart inaccessibility of the enzyme. Third, the formulation should be opaque to minimize the penetration of light into the formulation itself. It is worth noting that the light used for curing matrices has a small UV component, which could favor enzyme interchain polymerization or photo-oxidation. See Davies M J, supra. Fourth, excipients should not favor degradation or irreversible unfolding of enzymes. Finally, the formulation described herein was designed for a hypothetical case of very potent drug that needs to be released quickly.

The protected form was achieved by wet granulation with a 5% gelatin-B aqueous solution. The fundamental principle of wet granulation is to add a binder (e.g., gelatin aqueous solution) that will initially form liquid bridges between the particles (lactose and enzyme). See Remingtong J P, supra. These bridges allow the evolution of small aggregates and particles to larger entities. Further agglomeration of these entities results in the formation of a wet mass that can be granulated by sieving. Finally, gelation of gelatin confers strength to granules by holding together the components, which will then be dispersed within the gelatin gel. Therefore, granulation could be considered a macroencapsulation process. The rationale behind diluting the enzyme with a 100-fold excess of β-lactose was to decrease the probability of the enzyme residing on the outermost layers of granules and thus being available for interaction with the polymerizing species. Furthermore, the dilution step simulates a conventional pharmaceutical practice wherein a potent drug is diluted to avoid weighing errors. See Remingtong J P, supra; USP 24, supra. The choice of gelatin as a binder was based on the following considerations: it has a thermo-reversible gelation point around 37° C. This characteristic, in combination with the high solubility of β-lactose, allows granules to dissolve very rapidly when they come in contact with water or aqueous solutions maintained at 37° C. thereby affording intermediate availability of the entrapped molecules. See Kibbe A H, editor, “Handbook of pharmaceutical excipients,” 3^{rd} ed. Washington, D.C.: American Pharmaceutical Association, Pharmaceutical Press (2000); herein incorporated by reference. Nevertheless, because the amount of gelatin used for granulation was quite small (few drops of 5% gelatin-B aqueous solution per 1 g of unprotected powder), it was observed that granules dissolved in around 15 minutes even at 4° C.

Although granules were formulated with excipients that neither dissolved nor swelled in the monomer, solubility of formulated granules in monomeric PEGDM was investigated to exclude the possibility that granules could dissolve to some extent resulting in interactions between monomer and enzymes. Granules suspended in the monomeric PEGDM at room temperature, in the absence of light and photoinitiators, did not dissolve even after 2 days, and maintained their size, shape and opacity upon subsequent polymerization (FIG. 5). Furthermore, no leakage of enzyme from the granules was observed by optical microscopy (enzymes are colored) over the duration of contact with the monomer or during the polymerization step (FIG. 5). The absence of enzyme leakage from the granules may be attributed to the lack of solubility of β-lactose and gelatin in the monomer, and to the fact that the rate of diffusion of a molecule through a solid is negligible. Enzyme diffusion out from the granules into the monomer during the polymerization step, due to a possible increase in temperature, which could have melted the gelatin, may be excluded because the diffusion of a solid (the enzyme) in a rapidly solidifying environment (10-30 s) would be very difficult.

In the studies with 0.2 mm and 3 mm-thick matrices, we observed that the unprotected enzyme suffered a loss in activity upon entrapment. In addition, activity retention of unprotected enzymes, which was immobilized in thicker matrices, decreased with a decrease in salt content and an increase in CPH content. This trend may be due to an increase in the hydrophobicity of the system. Hydrophobic interactions are known to adversely affect protein structure. See Arakawa T, Prestrelski S J, Kenney W C, Carpenter J F, “Factors affecting short-term and long-term stabilities of proteins,” Adv Drug Del Rev 46, 307-326 (2001); herein incorporated by reference. The activity of protected enzymes appeared instead to be independent of both matrix composition and enzymatic characteristics, and therefore protection was considered successful.

MALDI-TOF analysis confirmed that changes in molecular weight of the unprotected enzymes do occur upon exposure to a photopolymerizing environment (Table 2). These changes in molecular weight correlate with a loss in enzyme activity in the case of both unprotected HRP and α-GLS. Such a loss in molecular weight is absent in the case of protected enzymes. This observation lends further credence to the hypothesis that reducing accessibility of a biomolecule can diminish the deleterious effects of the photopolymerizing environment.

Nevertheless, which component of the polymerizing environment caused the deactivation is not completely certain. Heat could have been a contributing factor. However, the decreased activity of unprotected HRP, which is thermostable, does not support this hypothesis. Light could have been another possible cause. However, native and formulated enzymes, when irradiated for 10 minutes in solid state or in an aqueous solution, in the presence of photoinitiators, maintained their activity, suggesting that light is an unlikely source of deactivation. Enzyme interactions with the monomer before the polymerization was not considered as a potential pathway for deactivation as the activity of the enzymes left in contact with the monomer for 2 minutes (see previous text) did not show variations (p>0.05). One could hypothesize that the loss of enzyme activity occurs during the diffusion process. However, the presence of lactose, which is known to have a stabilizing effect on proteins in aqueous solution, and the fast in vitro drug recovery, which aids in the retention of activity during the diffusion phase, suggest otherwise. Therefore, a likely cause of enzymatic deactivation may be interactions between monomers and drugs during the polymerization step.

What is claimed is:

1-81. (canceled)

82. A method, comprising:
exposing a composition to a photo-polymerizing environment, the composition comprising
a monomer capable of photo-polymerizing and cross-linking in the photo-polymerizing environment,
bioactive molecules,
a first material shielding the bioactive molecules from the photo-polymerizing environment, the first material being selected from the group consisting of gelatin, collagen, a natural polymer, and a synthetic polymer.

83. The method of claim 82, wherein the first material is gelatin.

84. The method of claim 82, wherein the first material comprises granulated particles.

85. The method of claim 82, wherein the first material shields the bioactive molecules from degradation caused by the photo-polymerizing environment.

86. The method of claim 82, wherein the first material is insoluble in the monomer.

87. The method of claim 82, wherein the first material is capable of transitioning from a solid to a gel at or above approximately a body temperature of a living organism.

88. The method of claim 82, wherein the bioactive material is capable of repairing or restructuring tissue of a living organism.

89. The method of claim 82, wherein the bioactive molecules comprise proteins.

90. The method of claim 82, wherein the composition further comprises a binder binding the first material and shielding the bioactive molecules from the photo-polymerizing environment.

91. The method of claim 90, wherein the binder comprises a sugar.

92. The method of claim 82 wherein the composition further comprises a plasticizer.

93. The method of claim 92, wherein the plasticizer comprises polyethylene glycol.

94. The method of claim 82, wherein the composition further comprises a disaggregant.

95. The method of claim 94, wherein the disaggregant comprises a cross-linked synthetic polymer.

96. The method of claim 82, wherein exposing the composition to the photo-polymerizing environment comprises polymerizing the monomer with visible radiation.

97. The method of claim 82, further comprising, after exposing the composition to the photo-polymerizing environment, placing the composition in an organism.
